(19) United States
(12) Patent Application Publication
(10) Pub. No.: US 2018/0200162 A1
(43) Pub. Date: Jul. 19, 2018

(54) O/W EMULSIONS

(71) Applicant: DSM IP ASSETS B.V., Heerlen (NL)

(72) Inventors: Christine MENDROK-EDINGER, Kaiseraugst (CH); Christine SAECKER, Kaiseraugst (CH); Karina HECKER, Kaiseraugst (CH)

(21) Appl. No.: 15/513,602

(22) PCT Filed: Sep. 22, 2015
(86) PCT No.: PCT/EP2015/071755

§ 371 Date: Mar. 23, 2017

(30) Foreign Application Priority Data
Sep. 26, 2014 (EP) 14186589.9

(51) Int. Cl. A61K 8/06; A61K 8/55; A61K 8/60; A61Q 19/00

(52) U.S. Cl. CPC A61K 8/062; A61K 8/55; A61K 2800/10; A61Q 19/00; A61K 8/602

(57) ABSTRACT

The present invention relates to topical compositions in the form of an oil-in-water (O/W) emulsion comprising an oil phase dispersed in an aqueous phase in the presence of a sucrose fatty ester surfactant, characterized in that the topical composition further comprises a phosphate ester co-surfactant. Furthermore, the invention relates to the use of a phosphate ester surfactant to reduce the viscosity of a topical composition in the form of an O/W emulsion comprising an oil phase dispersed in an aqueous phase in the presence of a sucrose fatty ester surfactant.

The present invention relates to topical compositions in the form of an oil-in-water (O/W) emulsion comprising an oil phase dispersed in an aqueous phase in the presence of a sucrose fatty ester surfactant, characterized in that the topical composition further comprises a phosphate ester co-surfactant. Furthermore, the invention relates to the use of a phosphate ester surfactant to reduce the viscosity of a topical composition in the form of an O/W emulsion comprising an oil phase dispersed in an aqueous phase in the presence of a sucrose fatty ester surfactant.

It is generally known that O/W emulsions with low viscosity often exhibit a stability problem upon storage, in particular at elevated temperatures. Thus, there is an ongoing need for agents that are able to reduce the viscosity O/W-emulsions without affecting the storage stability.

Surprisingly it has been found that the addition of a phosphate ester surfactant to a topical composition in the form of an oil-in-water (O/W) emulsion comprising an oil phase dispersed in an aqueous phase in the presence of a sucrose fatty ester surfactant results in a reduction of the viscosity without affecting the long term stability such as in particular the thermal stability.

Thus, in one embodiment, the present invention relates to topical compositions in the form of an oil-in-water (O/W) emulsion comprising an oil phase dispersed in an aqueous phase in the presence of a sucrose fatty ester surfactant, characterized in that the topical composition further comprises a phosphate ester co-surfactant.

In a further embodiment the invention relates to the use of a phosphate ester surfactant to reduce the viscosity of a topical composition in the form of an O/W emulsion comprising an oil phase dispersed in an aqueous phase in the presence of a sucrose fatty ester surfactant.

In another embodiment the invention relates to a method for reducing the viscosity of a topical composition in the form of an oil-in-water (O/W) emulsion comprising an oil phase dispersed in an aqueous phase in the presence of a sucrose fatty ester surfactant, said method comprising the addition of a phosphate ester co-surfactant.

In a further embodiment the invention relates to a method of preserving the long-term thermal storage stability of a topical composition in the form of an oil-in-water (O/W) emulsion comprising an oil phase dispersed in an aqueous phase in the presence of a sucrose fatty ester surfactant, said method comprising the addition of a phosphate ester co-surfactant. In a particular advantageous embodiment the topical composition exhibits an initial viscosity selected in the range of 1000 to 20′000 mPas, such as more preferably in the range of 2000 to 10′000 mPas (@ RT)

The amount of the sucrose fatty ester surfactant in the topical composition according to the present invention is preferably selected in the range of 0.1 to 10 wt.-%, preferably in the range of 0.3 to 5 wt.-%, most preferably in the range of 0.5 to 3 wt.-% based on the total weight of the topical composition.

The amount of the phosphate ester co-surfactant in the topical composition according to the present invention is preferably selected in the range of 0.01 to 3 wt.-%, preferably in the range of 0.05 to 2 wt.-%, most preferably in the range of 0.1 to 1 wt.-% based on the total weight of the topical composition.

Preferably in all embodiments of the invention the wt.-ratio between the sucrose fatty ester surfactant and the phosphate ester co-surfactant is additionally selected in the range of 8:1 to 1:1, preferably in the range of 6:1 to 2:1, most preferably in the range of 5:1 to 3:1 such as in the range of 4.5:1 to 3.5:1.

Particularly suitable in all embodiments of the invention are phosphate esters surfactants of formula (I)

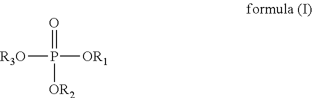

wherein
R₁, R₂ and R₃ are independently of each other hydrogen, C₁₋₂₂ alkyl, or a C₁₋₂₂ alkoxylated alkyl having 2 to 25 moles ethylene oxide,
with the proviso that at least one of R₁, R₂ and R₃ is an alkyl or an alkoxylated alkyl having at least 6 alkyl carbons in said alkyl or alkoxylated alkyl group.

Preferably R₁, R₂ and R₃ are independently of each other hydrogen, C₁₂₋₁₈ alkyl, or a C₁₂₋₂₈ alkoxylated alkyl having 2 to 12 moles ethylene oxide,

Monoesters in which R₁ and R₂ are hydrogen and R₃ is selected from C₁₂₋₁₈ alkyl and alkoxylated fatty alcohols having from 10 to 18 carbons and 2 to 12 moles ethylene oxide are preferred. Among the preferred phosphate ester surfactants are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, Dicetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium Cetyl Phosphate, Deceth-4 Phosphate, Deceth-6 Phosphate and Trilaureth-4 Phosphate. Most preferably, in all embodiments of the present invention, potassium cetyl phosphate e.g. commercially available as AMPHISOL® K at DSM Nutritional Products Ltd Kaiseraugst is used.

The term ‘sucrose fatty ester’ as used according to the present invention refers to compounds which are obtainable by esterification of fatty acids to one or several hydroxyl groups of sucrose. These esters are preferably chosen from mono-, di-, tri- and tetraesters, polyesters and their mixtures. Such sucrose fatty esters are widely used as surfactants in the cosmetic industry and well known to a person skilled in the art.

The term ‘fatty’, as used herein, preferably refers to a hydrocarbon chain having 12-22 carbon atoms (C₁₂, C₁₃, C₁₄, C₁₅, C₁₆, C₁₇, C₁₈, C₁₉, C₂₀, C₂₁, and C₂₂ in any sub-range or combination).

The chain may be straight or branched and may be saturated or unsaturated (typically one or two double bonds in the chain). Preferred fatty residues are chosen from stearates, behenates, cocoates, arachidonates, palmitates, myristates, laurates, carprates, oleates, laurates and their mixtures.

Preferable sucrose fatty esters according to the invention include sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate.

Most preferred sucrose fatty esters in all embodiments of the present invention are sucrose laurate (CAS 25339-99-5) e.g. sold under the tradename Surfhope SE Cosme C-1216 by Mitsubiushi, sucrose polystearate (CAS 37318-31-3 generic) e.g. sold under the name tradename Sisterna SP10-C or Sisterna SP01-C by Sisterna BV; sucrose stearate (CAS 37318-31-3, 25168-73-4) e.g. sold under the name tradename Sisterna SP70-C by Sisterna BV or under the name Surfhope SE Cosme C-1811; by Mitsubishi as well as mixtures thereof or with other substances such as Emulgade Sucro (BASF), a blend of sucrose polystearate and hydrogenated polyisobutene. Advantageously, in all embodiments of the invention the sucrose fatty ester is selected from the group consisting of sucrose laurate and sucrose stearate as well as mixtures thereof. Most preferably the sucrose fatty ester is sucrose stearate.

In a particular preferred embodiment the topical compositions according to the present invention contain—next to the sucrose fatty ester surfactant(s) and the phosphate esters co-surfactant(s)—additional surfactants, and/or co-surfactants, most preferably such additional surfactants, and/or co-surfactants are selected from the group consisting of fatty alcohols such as cetyl alcohol, stearyl alcohol, cetearyl alcohol, behenyl alcohol and the like and glyceryl stearate.

In another particular embodiment, the topical compositions according to the present invention are free of alkyl benzoate mixtures, wherein the sum of the C12- and C14-alkyl benzoates is greater than or equal to 85%, in relation to the total sum of the alkyl benzoates.

The viscosity reduction achieved by the phosphate ester surfactant is preferably at least 5%, more preferably at least 10% and most preferably in the range of about 30% to 50% measured at 20° C. [in mPas at 22° C.; Brookfield Viscosimeter DV-II Pro].

Particular preferred topical compositions according to the present invention exhibit a viscosity of 50′000 mPas or less, such as preferably a viscosity selected in the range of 100 to 30′000 mPas and most preferably selected in the range of 100 to 25′000 mPas after 10 days of storage at RT.

Most preferred are topical compositions according to the present invention which exhibit a viscosity of selected in the range of 250 to 25′000 mPas after 10 days of storage at RT, more preferably of 400 to 20′000 mPas and most preferably of 400-10′000 mPas such as e.g. in the range of 400 to 1000 mPas after 10 days of storage at RT.

The topical compositions according to the present invention can be in the form of a serum, milk, emulsion spray, lotion or cream, and they are prepared according to the usual methods. The compositions which are the subject-matter of the present invention are intended for topical application and can in particular constitute dermatological or cosmetic compositions which are, for example, intended for the protection of human skin against the adverse effects of UV radiation (antiwrinkle, anti-ageing, moisturizing, anti-sun protection and the like) or intended to moisturize and soothe the skin.

According to an advantageous embodiment of the invention the compositions constitute cosmetic composition which are intended for topical application to the skin.

The topical compositions of the invention preferably further contain usual cosmetic adjuvants and additives such as preservatives/antioxidants, fatty substances/oils, organic solvents, silicones, thickeners, softeners, emulsifiers, antifoaming agents, aesthetic components such as fragrances, surfactants, fillers, sequestering agents, anionic, cationic, nonionic or amphoteric polymers, propellants, acidifying or basifying agents, dyes, colorants, abrasives, absorbents, essential oils, sensory modifiers, astringents, or any other ingredients usually formulated into cosmetic compositions. Such cosmetic adjuvants and additives commonly used in the skin care industry, which are suitable for use in the compositions of the present invention are e.g. described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

In accordance with the present invention, the topical compositions according to the invention may further comprise one or more skin active or protective agent such as skin lightening agents, agents for tanning prevention and/or treatment of hyperpigmentation; skin-tanning agents; agents for the prevention or reduction of acne, anti-ageing agents, anti-inflammatory agents; moisturizers; anti-cellulite agents, slimming agents (e.g. phytanic acid), soothing agents, agents to improve the skin elasticity and skin barrier as well as UV-filter substances. The necessary amounts of such skin active or protective agents can, based on the desired product, easily be determined by the skilled person. The additional agents can either be added to the oil phase, the aqueous phase or separately as deemed appropriate. The mode of addition can easily be adapted by a person skilled in the art.

Particular suitable skin-lightening, tanning prevention and/or treatment of hyperpigmentation agents are in particular ascorbic acid and its derivatives such as sodium ascorbyl glycoside, sodium ascorbyl phosphate and magnesium ascorbyl phosphate, biotin, niacinamide, and/or alpha arbutin.

Particular suitable skin-tanning agents are dihydroxyacetone and/or erythrulose.

Particular suitable anti-ageing agents encompass natural extracts or peptides such as in particular SYN®-COLL or SYN®-AKE which are commercially available at DSM Nutritional Products Ltd.

Particular suitable UV-filter substances to be incorporated into the topical compositions according to the present invention encompass in particular the commercially available and widely used UV-filter substances octocrylene (PARSOL® 340), 4-methyl benzylidene camphor (PARSOL® 5000), ethylhexyl methoxycinnamate (PARSOL® MCX), ethylhexyl triazone (Uvinul® T-150), diethylhexyl butamido triazone (Uvasorb® HEB), 2,2′-methylene-bis-(6-(2H-benzotriazole-2-yl)-4-(1,1,3,3,-tetramethylbutyl)-phenol (Tinosorb® M), bis-ethylhexyl-oxyphenol methoxyphenyl triazine (Tinosorb® S), 2,2-(1,4-phenylene)bis-(1H-benzimidazol-4,6-disulfonic acid (NeoHeliopan® AP), 2-(4-Diethylamino-2-hydroxy-benzoyl)-benzoic acid hexylester (Uvinul® A plus), 1,1′-(1,4-piperazinediyl)bis[1-[2-[4-(diethylamino)-2-hydroxybenzoyl]phenyl]-methanone (CAS No 919803-06-8), polysilicone-15 (PARSOL® SLX), 2-phenyl benzimidazole sulfonic acid (PARSOL® HS), ethylhexyl salicylate (PARSOL® EHS), homomenthyl salicylate (PARSOL® HMS), Benzophenone-3 (Uvinul® M 40), Benzophenone-4 (Uvinul® MS 40), Butyl Methoxydibenzoyl Methane (PARSOL® 1789), Terephtalidene dicampher Sulfonic Acid (Mexoryl® SX), Drometrizole Trisiloxane (Mexoryl® XL), Tris-Biphenyl Triazine (nano) (Tinosorb® A2B), microfine zinc oxide (coated or uncoated) or titanium dioxide (coated or uncoated such as in particular PARSOL® TX as well as mixtures thereof.

Generally, the amount of each UV-filter substance in the topical compositions according to the invention is selected in the range of about 0.1 to 15 wt.-%, preferably in the range of about 0.5 to 10 wt.-%, most preferably in the range of about 1 to 8 wt.-% with respect to the total weigh of the topical composition.

The total amount of UV-filter substances in the topical compositions according to the invention is preferably selected in the range from 0.1 to 35 wt.-%, preferably in the range from 1 to 15 wt.-%, most preferably in the range from 5 to 35 wt.-% based on the total weight of the topical composition.

The cosmetically active ingredients useful herein can in some instances provide more than one benefit or operate via more than one mode of action.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 more preferably a pH in the range of 4.5 to 7.5 and most preferably in the range of 6 to 7.5. The pH can easily be adjusted as desired with suitable acids such as e.g. citric acid or bases such as sodium hydroxide (e.g. as aqueous solution), potassium hydroxide, triethanolamine (TEA Care), tromethamine (Trizma Base) and aminomethyl propanol (AMP-Ultra PC 2000) according to standard methods in the art.

The amount of the topical composition to be applied to the skin is not critical and can easily be adjusted by a person skilled in the art. Preferably the amount is selected in the range of 0.1-3 mg/cm² skin, such as preferably in the range of 0.1 to 2 mg/cm² skin and most preferably in the range of 0.5 to 2 wt.-%/cm².

The following examples are provided to further illustrate the compositions and effects of the present invention. These examples are illustrative only and are not intended to limit the scope of the invention in any way.

### EXAMPLE

The cosmetic formulations have been prepared according standard procedures.

Dissolve the water soluble ingredients in water and heat it up to 80° C. Combine the oil soluble ingredients and heat it up to 80° C. Combine the two phases under stirring and homogenize with an Ultra Turrax for 1 minute at 13000 rpm. Cool down under stirring. Add preservative and neutralizing agent below 40° C. under stirring.

The initial viscosity of a 100 g sample of each cream has been measured at 20° C. with a Brookfield Viscosimeter DV-II Pro in a 100 ml beaker 24 hours after preparation of the formulation using spindle S5 at 2.5 rpm.

The formulations are stored at RT (about 22° C.) and 40° C. The viscosities have consecutively been measured again as indicated in table 1.

The pH has been adjusted in the range of 6-7.5.

The results in table 1 illustrate that in the presence of potassium cetyl phosphate as co-surfactant the viscosity surprisingly increases during storage at 40° C. (compared to storage at RT), which indicates an excellent thermal stability. In contrast, in the absence of potassium cetyl phosphate (control) the viscosity decreases during storage at 40° C. (compared to storage at RT) as expected by a person skilled in the art which indicates the thermal instability. Furthermore, the formulation w/o potassium cetyl phosphate exhibits after 10 days an increased droplet size which is also an instability indicator.

TABLE 1

| | | | C |
|---|---|---|---|
| | A | B | control |
| | Wt.-% | Wt.-% | Wt.-% |
| Water | Ad 100 | Ad 100 | Ad 100 |
| Glycerin | 5.00 | 5.00 | 5.00 |
| Disodium EDTA | 0.10 | 0.10 | 0.10 |
| Xanthan Gum | 0.15 | 0.15 | 0.15 |
| Potassium Cetyl Phosphate | 0.50 | 0.50 | — |
| Butyl Methoxydibenzoylmethane | 4.00 | 4.00 | 4.00 |
| Octocrylene | 10.00 | 10.00 | 10.00 |
| Homosalate | 10.00 | 10.00 | 10.00 |
| Titanium Dioxide, Silica, Dimethicone | 3.00 | 3.00 | 3.00 |
| Sucrose Stearate | 2.00 | 1.50 | 2.00 |
| Cetearyl Alcohol | 2.00 | 2.00 | 2.00 |
| Butylene Glycol Dicaprylate/Dicaprate | 4.00 | 4.00 | 4.00 |
| Dicaprylyl Carbonate | 3.00 | 3.00 | 3.00 |
| Phenoxyethanol,Ethylhexylglycerin | 1.00 | 1.00 | 1.00 |
| Tromethamine | 0.25 | 0.25 | 0.25 |
| viscosity [mPas]/initial/RT | 3840 | 7840 | 28000 |
| viscosity reduction versus control | −86% | −72% | |
| viscosity [mPas]/10 days/RT | 480 | 520 | 16320 |
| viscosity [mPas]/10 days/40° C. | 3840 | 4480 | 6720 |
| stability outlook (microscopic evaluation) | stable | stable | unstable |

1. Topical compositions in the form of an oil-in-water (O/W) emulsion comprising an oil phase dispersed in an aqueous phase in the presence of a sucrose fatty ester surfactant, wherein the topical composition further comprises a phosphate ester co-surfactant.

2. The topical composition according to claim 1, wherein the amount of the sucrose fatty ester surfactant is selected in the range of 0.1 to 10 wt.-%, preferably in the range of 0.3 to 5 wt.-%, most preferably in the range of 0.5 to 3 wt.-% based on the topical weight of the topical composition.

3. The topical composition according to claim 1, wherein the amount of the phosphate ester co-surfactant is selected in the range of 0.01 to 3 wt.-%, preferably in the range of 0.05 to 2 wt.-%, most preferably in the range of 0.1 to 1 wt.-% based on the topical weight of the topical composition.

4. The topical composition according to claim 1, wherein the wt.-ratio between the sucrose fatty ester surfactant and the phosphate ester cosurfactant is selected in the range of 8:1 to 1:1, preferably in the range of 6:1 to 2:1, most preferably in the range of 5:1 to 3:1.

5. The topical composition according to claim 1, wherein the sucrose fatty ester surfactant is obtainable by esterification of a fatty acid having 12-22 carbon atoms to one or several hydroxyl groups of sucrose.

6. The topical composition according to claim 1, wherein the phosphate ester surfactant is a compound of formula (I) 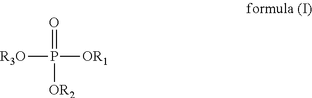
wherein
R₁, R₂ and R₃ are independently of each other hydrogen, C₁₋₂₂ alkyl, or a C₁₋₂₂ alkoxylated alkyl having 2 to 25 moles ethylene oxide, with the proviso that at least one of R₁, R₂ and R₃ is an alkyl or an alkoxylated alkyl having at least 6 alkyl carbons in said alkyl or alkoxylated alkyl group.

7. The topical composition according to claim 6, wherein the phosphate ester surfactant is potassium cetyl phosphate.

8. The topical composition according to claim 1, wherein the sucrose fatty ester is selected from the group consisting of sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate.

9. The topical composition according to claim 8, wherein the sucrose fatty ester is selected from the group consisting of sucrose laurate and sucrose stearate as well as mixtures thereof.

10. The topical composition according to claim 1, wherein the topical composition is in the form of a serum, an emulsion spray, a milk, a lotion or a cream.

11. Use of a phosphate ester surfactant to reduce the viscosity of a topical composition in the form of an O/W emulsion comprising an oil phase dispersed in an aqueous phase in the presence of a sucrose fatty ester surfactant.

12. A method for reducing the viscosity of a topical composition in the form of an oil-in-water (O/W) emulsion comprising an oil phase dispersed in an aqueous phase in the presence of a sucrose fatty ester surfactant, said method comprising the addition of a phosphate ester co-surfactant.

13. A method of preserving the long-term thermal storage stability of a topical composition in the form of an oil-in-water (O/W) emulsion comprising an oil phase dispersed in an aqueous phase in the presence of a sucrose fatty ester surfactant, said method comprising the addition of a phosphate ester co-surfactant.

14. The topical composition according to claim 1, wherein the topical composition is free of alkyl benzoate mixtures, wherein the sum of the C12- and C14-alkyl benzoates is greater than or equal to 85%, in relation to the total sum of the alkyl benzoates and exhibits a pH selected in the range of 6 to 7.5.

15. The topical composition according to claim 1, wherein the topical composition exhibits an initial viscosity in the range of 2000 to 10′000 mPas (@ RT).
